(19) United States
(12) Patent Application Publication
(10) Pub. No.: US 2023/0347000 A1
(43) Pub. Date: Nov. 2, 2023

(54) Portable Systems and Methods for Treating Materials with an Antimicrobial Agent

(71) Applicant: Applied Silver, Inc., Hayward, CA (US)

(72) Inventors: Austin Carey, Westlake Village, CA (US); Brandon Chadwell, Fremont, CA (US); Priya Balachandran, Palo Alto, CA (US); Elizabeth Hutt Pollard, Edmond, OK (US); Sean D. Morham, San Francisco, CA (US)

(21) Appl. No.: 17/800,657

(22) PCT Filed: Feb. 22, 2021
(86) PCT No.: PCT/US2021/019063

§ 371 Date: Aug. 18, 2022

Related U.S. Application Data
(60) Provisional application No. 62/979,175, filed on Feb. 20, 2020.

(51) Int. Cl. A61L 2/18; A61L 2/24

(52) U.S. Cl. CPC A61L 2/18; A61L 2/24; A61L 2202/11; A61L 2202/15; A61L 2202/16; A61L 2202/14; A61L 2202/26; A61L 2101/26

(57) ABSTRACT

A method for using a portable antimicrobial treatment system is provided. The method comprises: (i) providing water to a dilution reservoir, wherein a first flow controller controls a rate of flow of the water between a water supply and the dilution reservoir, (ii) providing a high ion concentrate to the dilution reservoir, wherein a dosing pump controls a flow rate of the ion concentrate between the metallic ion cartridge and the dilution reservoir; (iii) combining the water and the high ion concentrate into a diluted solution: (iv) providing the diluted solution to at least one application system, wherein a second flow controller control a rate of flow of the diluted solution between the dilution reservoir and the at least one application system; and (v) controlling, by an electronics control module, the first flow controller, the dosing pump, and the second flow controller.

### CROSS-REFERENCE TO RELATED APPLICATION

This application claims priority to U.S. Provisional Patent Application No. 62/979,175, filed on Feb. 20, 2020, which is hereby incorporated by reference in its entirety.

### FIELD

The disclosure relates to a portable system for storing and diluting concentrated materials and supplying diluted solutions of the concentrate to application equipment.

### BACKGROUND

The microbial contamination of all types of materials is often a threat to personal and public health. These materials may include liquids (e.g., water and/or other liquids), as well as other vicious materials (e.g., oils, gels, etc.) and soft surface materials, such as food products (e.g., meats, fruits, vegetables, cheeses, etc.), textiles (e.g., fabrics, carpets, curtains, and linens) In addition to health associated affects, the microbial contamination can contribute to unwanted characteristics of these materials, including odor and the spread of disease.

Accordingly, the inventors have identified a need in the art to provide portable, cost-effective, and convenient products and methods for applying antimicrobial products to soft surface materials and other surfaces in need thereof.

### SUMMARY

In one aspect, an example portable antimicrobial treatment system is disclosed. The portable antimicrobial treatment system comprises: (i) a water supply configured to provide water to an output of the water supply: (ii) a first flow controller connected to the output of the water supply; (iii) a metallic ion cartridge configured to provide a high ion concentrate to an output of the metallic ion cartridge; (iv) a dosing pump to dispense the high ion concentrate and connected to the output of the metallic ion cartridge; (v) a dilution reservoir connected to the output of the dosing pump and to the first flow controller, wherein the dilution reservoir is configured to combine the water and the high ion concentrate into a diluted solution: (vi) a second flow controller connected to an output of the dilution reservoir, wherein the provides the diluted solution to at least one application system; and (vi) an electronics control module connected to the first flow controller, the dosing pump, and the second flow controller.

In one aspect, a method for using a portable antimicrobial treatment system is provided. The method includes: (i) providing, by water supply, water to a dilution reservoir, wherein a first flow controller controls a rate of flow of the water between the water supply and the dilution reservoir; (ii) providing, by a metallic ion cartridge, a high ion concentrate to the dilution reservoir, wherein a dosing pump controls a flow rate of the ion concentrate between the metallic ion cartridge and the dilution reservoir: (iii) combining, by the dilution reservoir, the water and the high ion concentrate into a diluted solution: (iv) providing, by the dilution reservoir, the diluted solution to at least one application system, wherein a second flow controller controls a rate of flow of the diluted solution between the dilution reservoir and the at least one application system; and (v) controlling, by an electronics control module, the first flow controller, the dosing pump, and the second flow controller.

### BRIEF DESCRIPTION OF THE DRAWINGS

FIG. 1A shows a schematic representation of an example portable antimicrobial treatment system in a first state.

FIG. 1B shows a schematic representation of the example portable antimicrobial treatment system of FIG. 1A in a second state.

FIG. 2A shows an example portable antimicrobial treatment system in a first state.

FIG. 2B shows the example portable antimicrobial treatment system of FIG. 2A in a second state.

FIG. 2C shows the example portable antimicrobial treatment system of FIGS. 2A and 2B in a third state.

FIG. 3A is an exploded view of an example portion of the portable antimicrobial treatment system in a first state.

FIG. 3B is a representation of the example portion of the portable antimicrobial treatment system of FIG. 3A in a second state.

FIG. 4 is a flow chart showing an example process workflow for a portable antimicrobial treatment system.

FIG. 5 is a flow chart of an example method according to the disclosure.

### DESCRIPTION

The disclosure is directed to portable systems and methods for delivering diluted solutions of cleaning, deodorizing, antimicrobial, antistatic solutions and other useful solutions to all types of materials, but particularly liquids and soft surface materials. These materials include various liquids (e.g. water and/or other liquids), as well as other vicious materials (e.g., oils, gels, etc.) and soft surface materials, such as food products (e.g., meats, fruits, vegetables, cheeses, etc.), textiles (e.g., fabrics, carpets, curtains, and linens) and/or other materials where microbial contamination can contribute to unwanted characteristics of these materials, including odor and the spread of disease. The systems and methods may include also include dispensing such diluted solutions into various preexisting devices, such as a commercial or home-use textile washing machine. The portable systems (and methods for using the same) provide a supply of diluted solutions to the devices for delivery to a number of materials, and may do so in a number of ways.

In one aspect, the disclosure is directed to a portable, self-contained antimicrobial treatment system for storing a supply of a high ion concentrate solution that is diluted and delivered to various materials surfaces by a mechanisms and dispensing apparatuses contained within the portable antimicrobial treatment system. The portable antimicrobial treatment system may include a dilution reservoir for diluting the solution with a liquid diluent, such as water. Mechanical portions of the portable antimicrobial treatment system provide the appropriate pumps, controllers, and fluid communication between a water supply, antimicrobial agent chamber, and a dilution reservoir. The mechanical portion may also deliver diluted solution from the dilution reservoir to many different application systems.

In some aspects, the antimicrobial agent may be a metallic ion and the antimicrobial agent chamber may be a metallic ion cartridge may be replaceable or refillable. For instance, the metallic ion cartridge may include a leak-proof quick connection (e.g., a “single-click” installation) that provides fluid communication between the metallic ion cartridge and one or more of the mechanical portions or dilution reservoir of the portable antimicrobial treatment system. The quick connection allows for consumer replacement that avoids consumer contact with the liquid concentrate. In some aspects, the metallic ion cartridge may be refillable by the consumer, manufacturer, or other supplier.

The metallic ion cartridge, the dilution reservoir, and the mechanical portions of the portable antimicrobial treatment system each include the appropriate inlets and outlets for supplying liquid diluent and liquid concentrate to the dilution reservoir, and for supplying the diluted liquid to the dispensing apparatus (e.g., food processing equipment, one or more commercial washing machines, etc.).

The concentrate solution within the metallic ion cartridge includes a metallic ion, such as silver ion or copper ion, which are known for their deodorizing, antimicrobial and antistatic properties when applied to materials-particularly soft suface materials. In addition, the concentrate solution may contain cleaning or deodorizing compounds appropriate for the materials to which they are applied.

The liquid diluent may be water or another liquid appropriate for diluting the concentrate solution. The liquid diluent may be drawn into the portable antimicrobial treatment system from a separate diluent reservoir and/or from a continuous diluent supply line (e.g., an incoming water line, a water container, etc.).

The portable antimicrobial treatment system may include a housing, for example a rigid protective housing for confining the metallic ion cartridge, the dilution reservoir, and the mechanical portions of the portable antimicrobial treatment system.

In another aspect, the portable antimicrobial treatment system may include one or more mechanical portions that dispense diluted solution from the dilution reservoir to one or more materials, and may do so in a number of ways. For instance, the diluted solution may be delivered directly to one or more washing machines that include a wash basin for applying the diluted solution to textiles in the basin, usually combined with a level of wash or rinse water in the basin. The portable antimicrobial treatment system may also include one or more spraying mechanisms, vaporizers, or other such mechanisms that can apply the diluted solution to materials directly (e.g., spray directly on food products, such as meat, and % or on textile surfaces).

In some aspects, the portable antimicrobial treatment system may also include one or electronics control modules that control various aspects of the system. For example, the portable antimicrobial treatment system may also include an electronics control module that controls the mechanical portions of the system (e.g., pumps, flow controllers, dispensing apparatuses, etc.), as well as informational and identification systems described in further detail below.

For example, the portable antimicrobial treatment system may also include one or more information systems to alert the user, the manufacturer, support entities, and/or other parties of the operational status of the system. For example, the portable antimicrobial treatment system may include one or more user interfaces, external bulbs/lights, buttons/switches, and the like, to alert the user and/or a technician as to the operational status of the system. The portable antimicrobial treatment system may also include one or more network interfaces (e.g., WiFi, NFC, etc.) to pass this operational status information to the user, manufacturer, support entities, and/or other parties.

The portable antimicrobial treatment system may also include one or more identification systems to allow the correct matching of the metallic ion cartridge to the portable antimicrobial treatment system to prevent consumer mistake and avoid counterfeiting. For instance, an radio frequency identification (RFID) reader and an appropriate display may be used for matching a cartridge with the apparatus and warning the consumer of the use of the incorrect cartridge. These identification systems may also be used to identify other characteristics of the cartridge's operational status in the antimicrobial treatment system (e.g., the amount of the high ion concentrate in the cartridge, including after the cartridge provides the high ion concentrate). This and other technologies (e.g., barcode, UPC, blockchain) could be used for these purposes and others (e.g., tracking materials that have been treated with the diluted solution both inside and outside of a treatment facility, thereby creating electronic records of the antimicrobial treatment that follows the treated material from treatment to end use).

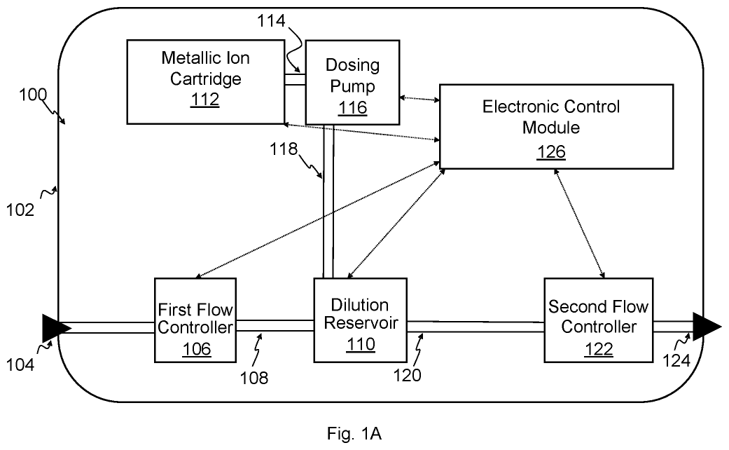

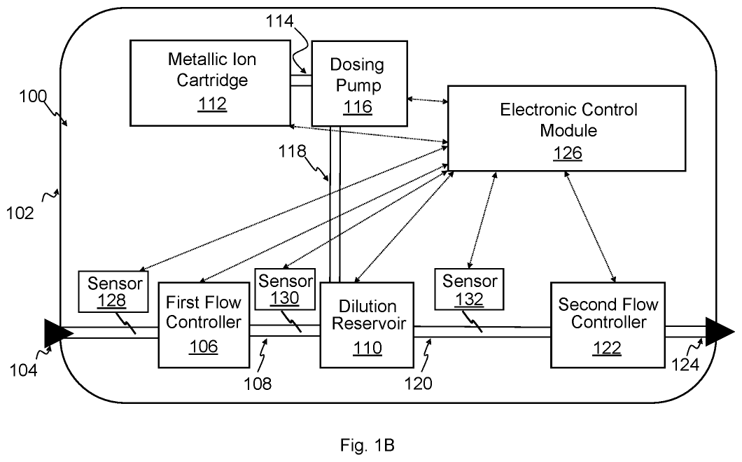

FIGS. 1A-B provide a schematic diagram of an example portable antimicrobial treatment system according to the disclosure. The features described herein can involve environments, operations, and functionalities that are configured or formatted differently, include additional or fewer components, include different types of components, and relate to one another in different ways. A portable antimicrobial treatment system 100 receives a liquid diluent (e.g., water), mixes the liquid diluent with a concentrate solution, and dispenses a diluted solution comprising, at least, the liquid diluent and the concentrate solution, all of which is self-contained and does not require major modification of existing systems and/or methods to treat materials.

In an example embodiment is illustrated in FIG. 1A. The system 100 may be made up of a housing 102, fluid connections (shown here as inlet 104, mechanical fluid connections 108, 114, 118, and 120, and outlet 124), a metallic ion cartridge 112, mechanical portions (shown here as first flow controller 106, dosing pump 116, and second flow controller 122), a dilution reservoir 110, and an electronics control module 126, which are represented in a particular configuration. FIG. 1A depicts only one example configuration, and other configurations are possible (and may even be preferable).

In this embodiment, the housing 102, fluid connections, and/or other components may be made of thermally-insulative and waterproof materials such as plastic, rubber, a combination of the two, but it should be understood that any of these components may be made of other materials. In a further aspect, the materials used to form any of these components may also be fortified or improved through one or more processes (e.g., chemical treatment).

In this embodiment, the inlet 104, the metallic ion cartridge 112, the outlet 124, and/or other components may be leak-proof quick connections that provide fluid communication between system and an apparatus with which the system is integrated. In a further aspect, because these components can encompass such quick connections, a consumer may replace or exchange various components within the system (or the entire systems itself) and avoid contact with any liquid concentrate solution inside the system. These quick connections may provide easy installation for the consumer, thereby obviating any need for professional installment.

Furthermore, the housing 102, the inlet 104, and/or the outlet 124 may also be modified to interact with a variety of connection types and configurations (e.g., existing food processing machinery, washing machines, washing machine drawers, dryers, vacuums, spraying devices, refrigerators, humidifiers, steamers, etc.), and the shape and/or dimensions of any component of system 100 can be modified to fit in or integrate with any device.

Turning to the operation of system 100, the system 100 receives a liquid diluent via inlet 104, the flow of which is controlled by first flow controller 106. First flow controller 106 (like the other mechanical portions illustrated in FIG. 1A) may be made up of one or more mechanical and/or electromechanical components (e.g., pumps, controllers, hydraulic valves, manual valves, solenoid valves, fluid regulators, and/or any other components used to facilitate regulated fluid communication), and may be controlled by mechanical forces (e.g. water pressure), electronic means (e.g., electronic control module 126), and/or both. First flow controller 106 may also supply dilution reservoir 110 via fluid connection 108.

The liquid diluent contained in the dilution reservoir 110 may be supplemented by a concentrate solution contained in the metallic ion cartridge 112 using dosing pump 116 and fluid connections 114 and 118. The concentrate solution in the metallic ion cartridge 112 may include a metallic ion, such as silver ion or copper ion, among other possibilities. In addition, the concentrate solution in the metallic ion cartridge 112 may contain cleaning or deodorizing compounds appropriate for the textiles to which they are applied. The metallic ion cartridge 112 may be replaced and/or refilled to provide an adequate supply of the appropriate concentrate solution.

Furthermore, the concentrate solution in the metallic ion cartridge 112 may be mixed with the liquid diluent in the dilution reservoir 110 by simply placing a liquid diluent and concentration in the dilution reservoir 110 and/or by using pumps and controllers (among other potential components) residing in the dilution reservoir 110 itself and/or other mechanical portions of the system. For example, the liquid diluent in the dilution reservoir 110 may be supplemented by concentrate solution contained in the metallic ion cartridge 112 by or more electrical pumps configured to provide fluid communication throughout the system 100 and may be contained in a mechanical portion such as first flow controller 106, dosing pump 116, and/or second flow controller 122.

Once mixed, the diluted solution may be transferred out of the dilution reservoir 110 and dispersed at one or more particular rates using second flow controller 122 via fluid connections 120 and 124. Additionally or alternatively, second flow controller 122 can also be used to dispense the diluted solution from the cartridge at a particular rate via fluid connections 120 and/or 124. In some examples, the mechanical portion may dispense the diluted solution using any number or configuration of mechanical and/or electromechanical components (e.g., pumps, controllers, hydraulic valves, manual valves, solenoid valves, fluid regulators, and/or any other components used to facilitate regulated fluid communication). Either way, any of these mechanical components may be in fluid communication with, but remain separate and distinct from, the dilution reservoir 110 and the metallic ion cartridge.

In a further aspect, the liquid diluent in the dilution reservoir 110 may be supplemented by concentrate solution contained in the metallic ion cartridge 112 and/or dispersed outside of system 100 based on one or more events. For example, the concentrate solution may be added to the liquid diluent and dispersed outside of system 100 in response to a particular event (e.g, at the onset of a new wash cycle in a washing machine), continuously (e.g., to allow an assembly line of food products to be consistently sprayed with diluted solution), periodically, and/or all of the above, among other possibilities.

Additionally, the mechanical portions of system 100 (and any components contained therein or in communication therewith) may be integrated throughout the system 100 as shown in FIG. 1A, among other possibilities. For example, an additional pump may be added between dilution reservoir 110 and second flow controller 122. In this way, dilution reservoir 110 and second flow controller 122 may have an intermediate mechanical portion to help mix the diluted solution, actively transferring diluted solution from the dilution reservoir 110 and second flow controller 122, and other such possibilities (e.g., by using a mechanical and/or electromechanical mixing component extending from an intermediate pump back into the dilution reservoir 110, by creating pressure and/or fluid volatility in the fluid connection between the components, etc.).

System 100 also contains an electronics control module 126, which may be connected to and/or control one or more of the dilution reservoir 110, the metallic ion cartridge 112, and the mechanical portions (shown here as first flow controller 106, dosing pump 116, and second flow controller 122) of system 100. Electronics control module 126 may also contain technology may also be used to allow the system 100 to be controlled and/or communicate with a variety of entities (e.g., the treating applications, a user, technician, off-site support entity, one or more servers, etc.), and may do so in a number of ways.

For example, the electronics control module 126 may be enabled with one or more technologies to allow the system 100 to be controlled and/or communicate with a variety of entities wired communication (e.g., USB. Mini-USB, and/or Lightning connections) and/or wireless communication (e.g., via Bluetooth. Near Field Communication. WI-FI, and/or other wireless connections), among other possibilities.

In this embodiment, monitoring and providing consistent concentration of the diluted solution may be advantageous and may be accomplished in one or more ways.

For example, turning to FIG. 1B, inlet 104 and fluid connection 108 may contain one or more sensors to monitor the quality of the received liquid diluent, among other possibilities (e.g., the flow rate of the incoming liquid diluent). For example, sensors 128 and/or 130 may be water quality sensors, mechanical and/or electronic rotameters, pressure sensors, or any combination of these, among other possibilities. In this way, electronics control module 126 may monitor and coordinate the incoming liquid diluent to ensure that what ultimately reaches dilution reservoir 110 is the highest quality (and most correct amount) to prepare the optimum diluted solution. Similarly, electronics control module 126 may monitor and coordinate the incoming concentrate from metallic ion cartridge 112 to ensure that what ultimately reaches dilution reservoir 110 is the correct brand and amount of concentrate to prepare the optimum diluted solution.

In a further aspect, fluid connection 120 may contain one or more sensors to monitor the quality of the diluted concentration to be dispensed, the source that the diluted concentration should be dispensed to, and/or the rate at which it is dispensed, among other possibilities (e.g., the flow rate of the outgoing diluted solution). For example, sensor 132 may be a conductivity sensor to confirm the presence and/or amount of metallic ions in the diluted solution, among other possibilities. In a further example, if the diluted solution is confirmed for distribution by the electronics control module 126, electronics control module 126 may also use second flow controller 122 to disperse the diluted solution to one or more application systems.

For example, electronics control module 126 may use second flow controller 122 to pump the diluted solution out of the dilution reservoir and disperse the diluted solution to one or more application systems (e.g., one or more food product treatment lines) using a variety of mechanical and/or electromechanical components (e.g., one or more electromechanical solenoid valves, each of which is in fluid connection with a particular food product treatment line).

In this way, electronics control module 126 may monitor and coordinate the outgoing diluted solution to ensure that what ultimately reaches the application systems (and the products that are treated using those application systems) is the highest quality (and most correct amount) treatment available.

In yet other examples, if the concentration of concentrate solution exceeds the expected or desired concentration level, one or more actions can be taken by the components in system 100. For example, if concentration is too high, the electronics control module 126 may cause other components to decrease the amount of concentrate solution incoming from metallic ion cartridge 112 (e.g., by closing or reducing the fluid communication of the concentrated solution). Additionally or alternatively, electronics control module 126 may cause other components of the system 100 to increase the amount of liquid diluent in the dilution reservoir 110 (e.g., by increasing the fluid communication of liquid diluent), thereby also lowering the concentration of concentrate solution in the dilution reservoir 110.

If, on the other hand, the concentration is lower than expected or desired, one or more actions can be taken by the components in the system 100. For example, if concentration is too low, electronics control module 126 may cause other components to increase the amount of concentrate solution incoming from the metallic ion cartridge 112 (e.g., by further opening or increasing the fluid communication of the incoming concentrated solution). Additionally or alternatively, the electronics control module 126 may cause other components of the system 100 to decrease the amount of liquid diluent in the dilution reservoir 110 (e.g., by decreasing or stopping the fluid communication of the liquid diluent), thereby also increasing the concentration of concentrate solution in the dilution reservoir 110.

In any event, the dilution reservoir 110 and the metallic ion cartridge 112 each include appropriate inlets and outlets for supplying liquid diluent and liquid concentrate solution to the dilution reservoir 110 via controlled and intelligent fluid communication, as well as for supplying the final diluted solution to a dispensing apparatus (e.g., food processing equipment, washing machine, etc.) via the outlet 124.

Additionally, system 100 may also include one or more informational systems to alert the user, the manufacturer, support entities, and/or other parties of the operational status of the system. For example, the system 100 may include one or more user interfaces, external bulbs/lights, buttons/switches, and the like, to alert the user and/or a technician as to the operational status of the system. These operations may allow the system to convey a variety of information (e.g., switching system 100 in enable/disable mode, option to place the machine in self-diagnostic “test mode.” etc.). As described above, the electronics control module 126 may also be connected to and/or control one or more of the informational systems, as well as technologies that allow the system 100 to be controlled and/or communicate with a variety of entities (e.g., a user, technician, off-site support entity, one or more servers, etc.) via wired and/or wireless communication, among other possibilities. The electronics control module 126 may also include one or more network interfaces to pass this operational status information to the user, manufacturer, support entities, and/or other parties.

Additionally, the system 100 may also include one or more identification systems to allow the correct matching of the metallic ion cartridge 112 to the system 100 to prevent consumer mistake and avoid counterfeiting. For instance, an RFID reader and an appropriate display may be used for matching the metallic ion cartridge 112 to the system 100 and waring the consumer of the use of the incorrect cartridge. These identification systems may also be used to identify other characteristics of metallic ion cartridge's and/or the system's operational status (e.g., the amount of concentrate in metallic ion cartridge 112, including after the metallic ion cartridge 112 provides the high ion concentrate to the dilution reservoir 110).

These identification systems may also be used to identify characteristics of materials to be treated (e.g., electronics control module may detect an RFID tag on a food product that indicates the food product has already been treated with a sufficient antimicrobial and should not be treated again). In this way, RFID may be coupled to or associated with materials to be treated. In an example, the RFID tag can include an integrated circuit (IC) chip that stores the identification information associated with the material. The RFID tag can further include an antenna for communicating with the electronics control module 126 and/or a protective housing for protecting the RFID during use treatment methods (e.g., processing). This protective housing can provide a waterproof, heat resistant, and % or pressure resistant enclosure. Within examples, the RFID technology described herein can be a passive RFID tag, a semi-passive RFID tag, and/or an active RFID tag.

In a further aspect, RFID and other technologies (e.g., barcode, UPC, blockchain) could be used for these purposes and others. For example, a treatment facility using the system 100 could implement an internal RFID application protocol where as soon as a material is treated, a tracking mechanism (e.g., an RFID tag, barcode, etc.) is affixed to the treated material (and/or associated packing materials) and an electronic record associated with the tracking mechanism can be created (e.g., by the electronics control module 126). Then, once this tracking mechanism and electronic record are created, these treated materials can be tracked both inside the facility (e.g., for inventory purposes) and outside of the facility (e.g., by linking the internal system tracking capabilities to outside systems to track treated lots of soft and/or viscous materials with barcode/RFID/blockchain or other supply chain-enabling technologies to the end-use point).

Turning back to the electronics control module 126, an example detailed operation of system 100 in connection with FIG. 1B is provided below. In an example operation, electronics control module 126 may receive a signal to begin mixing a diluted solution from a treatment application (e.g., a food product sprayer that the electronics control module 126 is connected with over WiFi). Upon receiving this signal, the electronics control module 126 opens an electromechanical solenoid valve in the first flow controller 106 and measures the quality of water coming in via the inlet 104 using the sensor 128 and the pressure of the incoming water in the fluid communication 108 using the sensor 130. Once the electronics control module 126 determines adequate water quality and flow rate, the electronics control module 126 instructs the dosing pump 116 to dispense concentrated solution from the metallic ion cartridge 112 into the dilution reservoir 110, while also monitoring the presence and amount of silver ions in diluted solution being mixed in the dilution reservoir 110 and the fluid communication 120 using the sensor 132. Once a predetermined concentration of silver ion is detected in the diluted solution, the electronics control module 126 instructs the dosing pump 116 to stop dispensing concentrated solution from the metallic ion cartridge 112. The electronics control module 126 then instructs the second flow controller 122 to pump the diluted solution out of the dilution reservoir 110 and to deliver the diluted solution to the one or more treatment applications by opening one or more electromechanical solenoid valves in the second flow controller 122. During this last step, the electronics control module 126 may instruct the electromechanical solenoid valve in first flow controller 106 to remain open (e.g., to flush the dilution reservoir 110, second flow controller 122, fluid connection 120, and/or outlet 124 with incoming liquid diluent) or to close, depending on the particular application.

The electronics control module 126 may also take further actions after the completion of this detailed operation, for example: (i) creating a timestamp of all of these events; (ii) log data associated with sensors 128, 130, and 132 (e.g., water quality data, flow rate data, concentration data); (iii) reporting any errors or other operational status errors to the user, a support entity, etc.: and/or (iv) updating an RFID tag on the metallic ion cartridge 112 to indicate the amount of the high ion concentrate after the metallic ion cartridge 112 provides the high ion concentrate, among other possibilities.

The electronics control module 126 may also take additional actions before, after, or agnostic of the completion of this detailed operation, for example: (i) constantly monitoring for input data from one or more application systems; (ii) constantly monitoring for input data from one or more users or support entities; and/or (iii) periodic and/or event based data uploads to one or more serves for system configuration changes, among other possibilities. Furthermore, the instructions that cause the electronics control module 126 to execute these detailed operational steps (and others), may be executed and/or updated locally and/or remotely (e.g., by using a JSON upload and/or script configuration download, locally and/or remotely, using wired and/or wired communications).

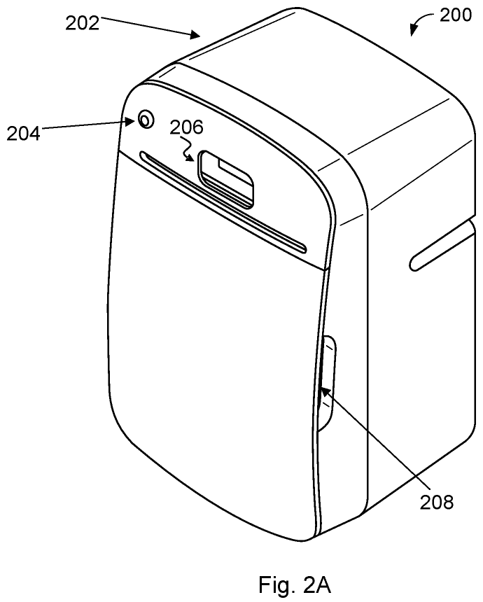

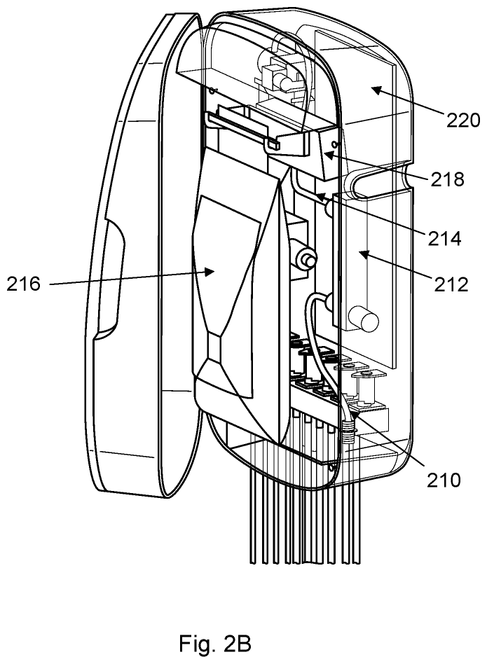

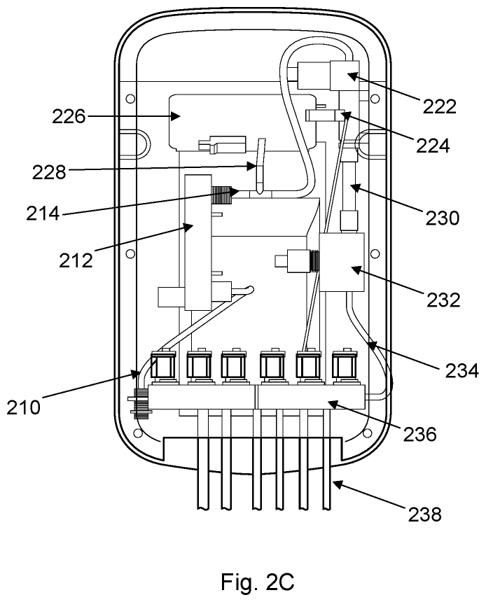

FIGS. 2A, 2B, 2C, 3A, 3B, and 4, show various views of other example embodiments using the same or similar components of the portable antimicrobial treatment systems depicted in FIGS. 1A-1B.

FIGS. 2A-C provide a schematic representation of an example portable antimicrobial treatment system according to the disclosure. The features described herein can involve environments, operations, and functionalities that are configured or formatted differently, include additional or fewer components, include different types of components, and relate to one another in different ways. A portable antimicrobial treatment system 200 receives a liquid diluent (e.g., water), mixes the liquid diluent with a concentrate solution, and dispenses a diluted solution comprising, at least, the liquid diluent and the concentrate solution, all of which is self-contained and does not require major modification of existing systems and/or methods to treat materials.

In an example embodiment, as illustrated in FIG. 2A, the system 200 is made up of a housing 202. The system 200 may have one or more power indicators 204 that may be used, among other things, indicating to a user/operator that the system 200 has sufficient power or is presently powered on or off, among other things. Although shown as small, round indicator in FIG. 2A, it should be readily understood that power indicator 204 may be a different shape and/or made of different components (e.g., a strip of multicolor LEDs, each color of which may indicate different operational statuses of the system 200).

The system 200 may also contain display 206 may also be used for displaying information to the user/operator (e.g., indicating a cartridge is matched with the system and/or warning the consumer of the use of the incorrect cartridge). The display 206 may also be used to display other characteristics of the operational status of the system 200 (e.g., the current operational status of the system 200 (e.g., “SETUP”, “RUNNING”, “ERROR”), the amount of the high ion concentrate in the cartridge, including after the cartridge provides the high ion concentrate during one or more treatment cycles).

The system 200 may also contain one or more access panels 208 (e.g., a door) that allow a user/operator, technician, and/or support entity to access the interior components of the system 200.

For example, turning to the operation of the system 200 as shown in FIG. 2B, the system 200 receives a liquid diluent via inlet 210, the flow of which is controlled by first flow controller 212. The first flow controller 212 (like the other mechanical portions illustrated in FIGS. 1A and 1B) may be made up of one or more mechanical and/or electromechanical components (e.g., pumps, controllers, hydraulic valves, manual valves, solenoid valves, fluid regulators, and/or any other components used to facilitate regulated fluid communication), and may be controlled by mechanical forces (e.g., water pressure), electronic means (e.g., an electronics control module), and/or both. The first flow controller 212 may also supply a dilution reservoir via fluid connection 214.

Additionally, as illustrated in FIG. 2B, like FIGS. 1A and 1B, the system 200 may also include one or more identification systems to allow the correct matching of the metallic ion cartridge 216 to the system 200 to prevent consumer mistake and avoid counterfeiting. For instance, an RFID reader 218 and an appropriate display (e.g., display 206) may be used for matching the metallic ion cartridge 216 to the system 200 and warning the consumer of the use of the incorrect cartridge. These identification systems may also be used to identify other characteristics of metallic ion cartridge's and/or the system's operational status (e.g., the amount and/or type of concentrate in metallic ion cartridge 216, including after the metallic ion cartridge 216 provides the high ion concentrate to the system 200).

For example, the system 200 may use the electronics control module 220 to operate and interpret input signals from the RFID reader 218 to identify operational characteristics of the metallic ion cartridge 216 and/or the system 200, among other possibilities. For example, the system 200 may also contain use the electronics control module 220 to monitor and/or control various components of the system 200 (including the power indicator 204, display 206, one or more portions of the access panel 208 (e.g., an electronic lock on access panel 208), the metallic ion cartridge 216, the RFD reader 218, and/or the mechanical portions of system 200 (e.g., the first flow controller 212).

The electronics control module 220 may also be used to allow the system 200 to be controlled and/or communicate with a variety of entities (e.g., the treating applications, a user, technician, off-site support, one or more servers, etc.), and may do so in a number of ways.

For example, the electronics control module 220 may include with one or more technologies to allow the system 200 to be controlled and/or communicate with a variety of wired communication (e.g., USB. Mini-USB, and/or Lightning connections) and/or wireless communication (e.g., via Bluetooth, Near Field Communication, WI-FI, and/or other wireless connections), among other possibilities.

In this embodiment, monitoring and providing consistent concentration of the diluted solution may be advantageous and may be accomplished in one or more ways.

For example, as shown in FIG. 2C, the system 200 receives a liquid diluent via the inlet 210, the flow of which is controlled by the first flow controller 212. The first flow controller 212 may also supply the dilution reservoir 222 via fluid connection 214.

The liquid diluent contained in the dilution reservoir 222 may be supplemented by a concentrate solution contained in the metallic ion cartridge 216 using dosing pump 226 and fluid connections 224 and 228. The concentrate solution in the metallic ion cartridge 216 may include a metallic ion, such as silver ion or copper ion, among other possibilities. In addition, the concentrate solution in the metallic ion cartridge 216 may contain cleaning or deodorizing compounds appropriate for the textiles to which they are applied. The metallic ion cartridge 216 may be replaced and/or refilled to provide an adequate supply of the appropriate concentrate solution.

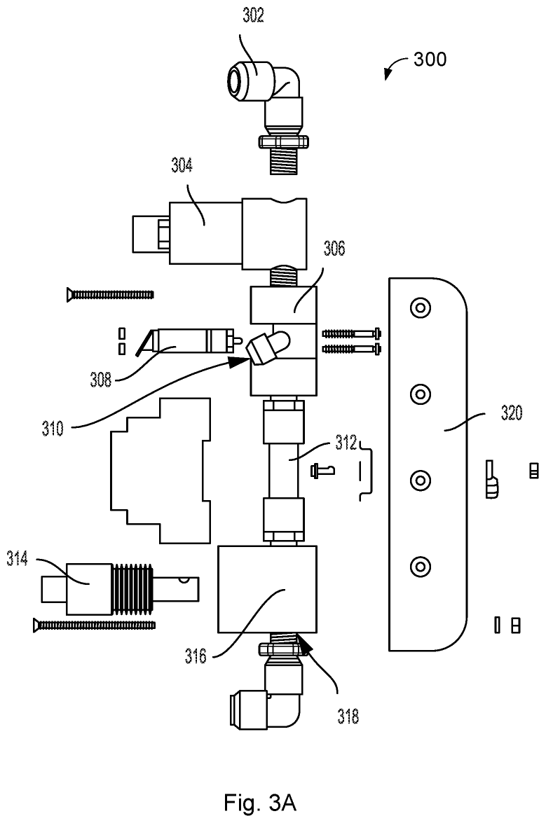

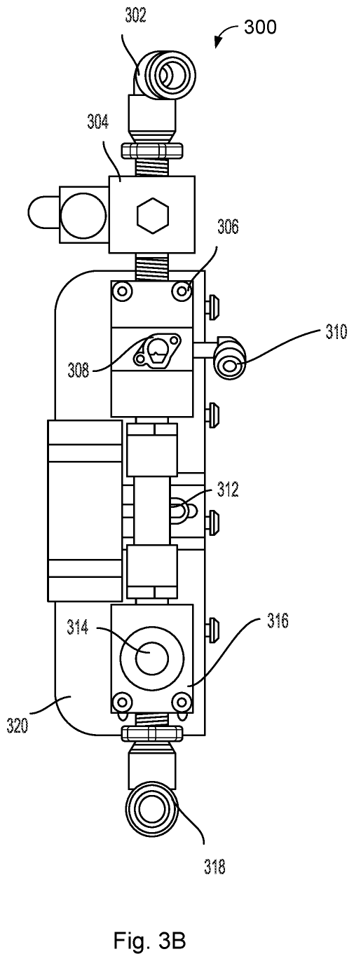

Furthermore, the concentrate solution in the metallic ion cartridge 216 may be mixed with the liquid diluent in the dilution reservoir 222 by simply placing a liquid diluent and concentration in the dilution reservoir 222 and/or by using pumps and controllers (among other potential components) residing in the dilution reservoir 222 itself and/or other mechanical portions of the system (for example, as further illustrated in FIGS. 3A and 3B, below).

Once mixed, the diluted solution may be transferred out of the dilution reservoir 222 and dispersed at one or more particular rates using second flow controller 236 via fluid connections 230 and 234, and outlet 238. Additionally or alternatively, the second flow controller 236 can also be used to dispense the diluted solution from the cartridge at a particular rate in response to a particular event (e.g., at the onset of a new wash cycle in a washing machine), continuously (e.g., to allow an assembly line of food products to be consistently sprayed with diluted solution), periodically, and/or all of the above, among other possibilities. Further, the system 200 may use one or more components to also ensure that the proper concentration of diluted solution is dispensed from the system 200.

For example, as shown in FIG. 2C, the system 200 may include one or more sensors 232. Sensor 232 may be a conductivity sensor to confirm the presence and/or amount of metallic ions in the diluted solution, among other possibilities. In a further example, if the diluted solution is confirmed for distribution by the electronics control module 220, the electronics control module 220 may also use the second flow controller 236 to disperse the diluted solution to one or more application systems.

For example, electronics control module 220 may use second flow controller 236 to pump the diluted solution out of the dilution reservoir and disperse the diluted solution to one or more application systems (e.g., one or more food product treatment lines) using a variety of mechanical and/or electromechanical components (e.g., one or more electromechanical solenoid valves, each of which is in fluid connection with a particular food product treatment line).

In this way, the electronics control module 220 may monitor and coordinate the outgoing diluted solution (e.g., using the sensor 232 and/or the second pump controller 236) to ensure that what ultimately reaches the application systems (and the products that are treated using those application systems) is the highest quality (and most correct amount) treatment available.

In yet other examples, if the concentration of concentrate solution exceeds the expected or desired concentration level, one or more actions can be taken by the components in the system 200.

For example, if concentration is too high (e.g., based on conductivity data detected by the sensor 232), the electronics control module 220 may cause other components to decrease the amount of concentrate solution incoming from the metallic ion cartridge 216 (e.g., by closing or reducing the fluid communication of the concentrated solution). Additionally or alternatively, the electronics control module 220 may cause other components of the system 200 to increase the amount of liquid diluent in the dilution reservoir 222 (e.g., by increasing the fluid communication of liquid diluent), thereby also lowering the concentration of concentrate solution in the dilution reservoir 222.

If, on the other hand, the concentration is lower than expected or desired (e.g. based on conductivity data detected by the sensor 232), one or more actions can be taken by the components in the system 200. For example, if concentration is too low, the electronics control module 220 may cause other components to increase the amount of concentrate solution incoming from the metallic ion cartridge 216 (e.g., by further opening or increasing the fluid communication of the incoming concentrated solution). Additionally or alternatively, the electronics control module 220 may cause other components of the system 200 to decrease the amount of liquid diluent in the dilution reservoir 222 (e.g., by decreasing or stopping the fluid communication of the liquid diluent), thereby also increasing the concentration of concentrate solution in the dilution reservoir 222. Other examples are possible.

FIGS. 3A-B provide views of an example portion of a portable antimicrobial treatment system according to the disclosure. The features described herein can involve environments, operations, and functionalities that are configured or formatted differently, include additional or fewer components, include different types of components, and relate to one another in different ways. Portable antimicrobial treatment system components 300 receive a liquid diluent (e.g., water), mixes the liquid diluent with a concentrate solution, measures one or more parameters associated with a diluted solution (e.g., conductivity), and passes the diluted solution via an outlet and does not require major modification of existing systems and/or methods to treat materials.

In an example embodiment, as illustrated in FIG. 3A, the components 300 may be made up of first inlet 302, first mechanical portion 304, dilution reservoir 306, second mechanical portion 308, second inlet 310, fluid connection 312, sensor 314, sensor chamber 316, outlet 318, and mounting plate 320, and are depicted in a particular configuration. FIG. 3A, however, depicts only one example configuration, and other configurations are possible (and may even be preferable).

In this embodiment, the individual components may be made of thermally-insulative and waterproof materials such as plastic, rubber, a combination of the two, but it should be understood that any of these components may be made of other materials, including precision machined metal components and other metals and metal alloys (e.g., aluminum, steel, etc.). In a further aspect, the materials used to form any of these components may also be fortified or improved through one or more processes (e.g., chemical treatment, anodized aluminum, etc.).

In this embodiment, the first inlet 302, the second inlet 310, the outlet 318, and/or other components may be leak-proof quick connections that provide fluid communication between system and an apparatus with which the system is integrated. In a further aspect, because these components can encompass such quick connections, a consumer may replace or exchange various components within the system (or the entire systems itself) and avoid contact with any liquid concentrate solution inside the system. These quick connections may provide easy installation for the consumer (e.g., by quick connections to the first inlet 302, the second inlet 310, and the outlet 318), thereby obviating any need for professional installment.

Furthermore, the first inlet 302, the second inlet 310, the outlet 318, and % or mounting plate 320 may also be modified to interact with a variety of connection types and configurations and the shape and/or dimensions of any component of the components 300 can be modified to fit in or integrate with any device.

Turning to the operation of the components 300, the components 300 receives a liquid diluent via the first inlet 302, the flow of which is controlled by the first mechanical portion 304 and/or the second mechanical portion 308, which may be made up of one or more mechanical and/or electromechanical components (e.g., pumps, controllers, hydraulic valves, manual valves, solenoid valves, fluid regulators, and/or any other components used to facilitate regulated fluid communication), and may be controlled by mechanical forces (e.g., water pressure), electronic means, and/or both. For example, the first mechanical portion 304 may be a valve (e.g., a solenoid valve) that opens to allow a diluent to enter the dilution reservoir 306.

The liquid diluent contained in the dilution reservoir 306 may also be supplemented by a concentrate solution (e.g. from a metallic ion cartridge) via the second inlet 310. Ina further aspect, once the diluent and concentrate solution are both in the dilution reservoir, the diluent and concentrate solution may be mixed in the dilution reservoir 306 by using second mechanical portion 308 (e.g., solenoid pumps and controllers (among other potential components)) residing in the dilution reservoir 306. For example, the liquid diluent in the dilution reservoir 306 may be supplemented by and/or mixed with the concentrate solution (via second inlet 310) and then transferred out of the dilution reservoir 306 via fluid connection 312. Additionally or alternatively, the fluid connection 312 may also have an intermediate mechanical portion to help mix the diluted solution (e.g., by using a static mixer contained inside the fluid connection 312, a mechanical and/or electromechanical mixing component extending into the fluid connection 312 from the first mechanical portion 304 and/or the second mechanical portion 308, etc.).

In a further aspect, as shown in FIG. 3A, once the diluted solution is transfened from the fluid connection 312, one or more parameters of the diluted solution may be detected and/or measured using the one or more sensors 314 residing in the sensor chamber 316 (which may contain one or more additional mechanical components controlling the release or flow rate for the diluted solution entering or exiting the sensor chamber 316). Sensor 314 may be a conductivity sensor to confirm the presence and/or amount of metallic ions in the diluted solution, among other possibilities (such as those described herein). In a further example, if the diluted solution is confirmed for distribution (e.g., by an electronics control module), the diluted solution may be dispensed to one or more application systems via the outlet 318.

Finally, as shown in FIG. 3A, the system components 300 may be mounted to antimicrobial treatment systems, at initial manufacturing and/or added to existing systems using mounting plate 320, which may not require major modification of existing systems and/or methods to treat materials.

Turning to FIG. 3B. FIG. 3B provides a representation of the example portion of a portable antimicrobial treatment system show in FIG. 3A, except fully assembled and from a different view. As illustrated in FIG. 3B, the components 300 may be made up of the first inlet 302, the first mechanical portion 304, the dilution reservoir 306, the second mechanical portion 308, the second inlet 310, the fluid connection 312, the sensor 314, the sensor chamber 316, the outlet 318, and the mounting plate 320, and are depicted in a particular configuration. Other configurations are possible.

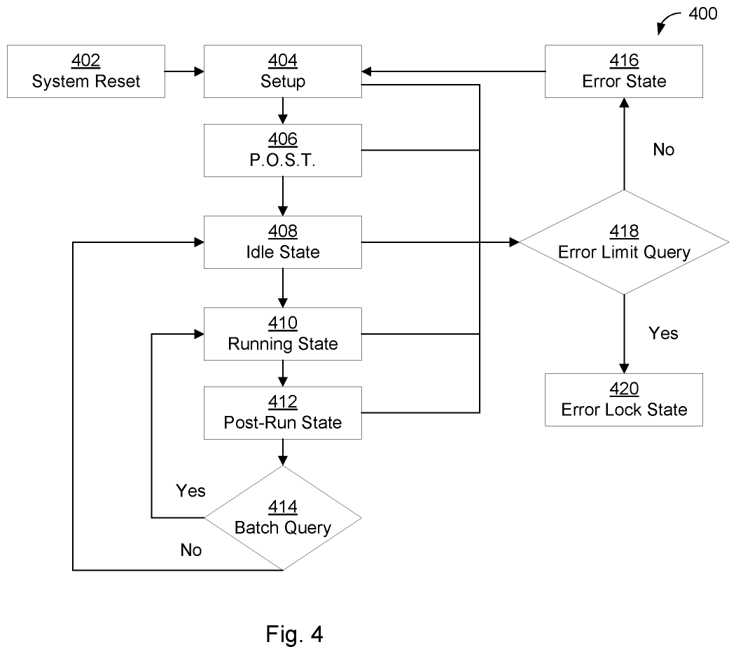

FIG. 4 shows an example process workflow for a portable antimicrobial treatment system (e.g., the system 100 and/or the system 200). The features described herein can involve environments, operations, and functionalities that are configured or formatted differently, include additional or fewer steps and/or computational actions, include different types of steps and/or computational actions, and relate to one another in different ways.

As described above, a portable antimicrobial treatment system may contain an electronics control module, which may be used to receive data (e.g., from user input, sensor data, etc.), make one or more determinations, and, in response, control one or more components of the portable antimicrobial system (e.g., one or more mechanical and/or electromechanical components of the portable antimicrobial system). To do so, the electronics control module may execute one or more sets of program instructions and use one or more components to facilitate this process.

For example, the electronics control module can be configured to perform and/or can perform one or more acts and/or functions, such as those described in this disclosure. The electronics control module can include various components, such as one or more processors, a data storage unit, a communication interface, and/or a user interface. Each of these components can be connected to each other via a connection mechanism. In this disclosure, the term “connection mechanism” means a mechanism that facilitates communication between two or more components, devices, systems, or other entities. A connection mechanism can be a relatively simple mechanism, such as a cable or system bus, or a relatively complex mechanism, such as a packet-based communication network (e.g., the Internet). In some instances, a connection mechanism can include a non-tangible medium (e.g., in the case where the connection is wireless).

The one or more processors can include a general-purpose processor (e.g., a microprocessor) and/or a special-purpose processor (e.g., a digital signal processor (DSP)). The one or more processors can execute program instructions included in the data storage unit, as discussed further below.

The data storage unit can include one or more volatile, non-volatile, removable, and/or non-removable storage components, such as magnetic, optical, and/or flash storage, and/or can be integrated in whole or in part with the processor. Further, the data storage unit can take the form of a non-transitory computer-readable storage medium, having stored thereon program instructions (e.g., compiled or non-compiled program logic and/or machine code) that, upon execution by the processor, cause the electronics control module to perform one or more acts and/or functions, such as those described in this disclosure. These program instructions can define, and/or be part of, a discrete software application. In some instances, the electronics control module can execute program instructions in response to receiving an input, such as an input received via the communication interface and/or the user interface. The data storage unit can also store other types of data, such as those types described in this disclosure.

The communication interface of the electronics control module can allow the electronics control module to connect with and/or communicate with another entity, such as another computing device, according to one or more protocols. In one example, the communication interface can be a wired interface, such as an Ethernet interface. In another example, the communication interface can be a wireless interface, such as a celhlar or WI FI interface. In this disclosure, a connection can be a direct connection or an indirect connection, the latter being a connection that passes through and/or traverses one or more entities, such as a router, switch, or other network device. Likewise, in this disclosure, a transmission can be a direct transmission or an indirect transmission.

The user interface can include hardware and/or software components that facilitate interaction between electronics control module and a user of the portable antimicrobial system, if applicable. As such, the user interface can include input components such as: the display described herein (e.g., in connection with FIG. 2A), a keyboard, a keypad, a mouse, a touch sensitive panel, and/or a microphone, and/or output components such as a display device (which, for example, can be combined with a touch sensitive panel), a sound speaker, and/or a haptic feedback system.

In a further aspect, the features described herein may involve some or all of these components arranged in different ways, including additional or fewer components and/or different types of components, among other possibilities.

As represented in FIG. 4, the electronics control module may execute program instructions to reflect various operational states of the portable antimicrobial device in order to perform various operations and process workflow 400 described herein, some of which will be described in further detail below.

In FIG. 4, the electronics control module may begin with a system reset 402. Once reset, the electronics control module may execute program instruction to begin a setup protocol 404. During setup 404, electronics control module may initialize one or more components of the portable antimicrobial system (e.g., one or more sensors, mechanical and/or electromechanical components of the system).

After setup 404, the electronics control module may execute program instruction to begin a Power On Self Test (“P.O.S.T.”) protocol 406. During P.O.S.T., the electronics control module may runs through a series of component tests to ensure the system is running properly and/or ensure that one or more components of the portable antimicrobial system (e.g., one or more sensors, mechanical and/or electromechanical components of the system) are performing to one or more predetermined specifications.

If, during the P.O.S.T. protocol 406, the electronics control module determines that one or more components of the system are not performing up to a predetermined specification, the electronics control module may take one or more responsive actions. For example, if the electronics control module determines that one of the sensors of the system is not functioning properly, the electronics control module may determine that the system may not proceed with supplying an antimicrobial treatment until the sensor is repaired and/or cause the user interface/display of the system to display an error message reflecting the error. In another example, the electronics control module may determine that the RFID tag on a cartridge is not matched with the system and/or warn the consumer of the use of the incorrect cartridge via the user interface and/or display. Other examples are possible.

If, during the P.O.S.T. protocol 406, the electronics control module determines that one or more components of the system are performing up to a predetermined specification, the electronics control module may proceed to idle state 408. In idle state 408, the electronics control module has determined that the one or more components of the system are performing up to a predetermined specification and that the system is ready to proceed to a treatment cycle. From the idle state 408, the electronics control module may automatically proceed to a treatment cycle (e.g., based on predetermined amount of time passing) and/or based on user/operator input (e.g., a request from a user to initiate a treatment cycle, a request from a washer to initiate a treatment cycle, etc.), among other possibilities.

In any event, once the electronics control module determines that a treatment cycle should be initiated, it may proceed to running state 410, which indicates that the system is performing a treatment cycle, which is described in detail in connection with, for example, the figures and accompanying disclosure herein.

Once the electronics control module determines that the treatment cycle is complete, it may proceed to post-run state 412, which indicates that the system has performed a treatment cycle, and may undertake one or more actions thereafter. For example, in the post-run state 412, the electronics control module may perform one or more actions to update records and/or components associated with the system.

For example, in the post-run state 412, the electronics control module may update the RFID tag on a cartridge to reflect the amount of concentrated solution that was removed from the cartridge during the treatment cycle and/or inform the consumer of the amount of concentrated solution remaining in the cartridge via the user interface and/or display. In another example, the electronics control module may generate one or more reports summarizing the results and/or component details during the treatment cycle). In this way, as described above, the electronics control module may also be connected to and/or control one or more of the informational systems, as well as technologies that allow the system to communicate with a variety of entities (e.g., a user, technician, off-site support entity, one or more servers, etc.) to pass this operational status information to the user, manufacturer, support entities, and/or other parties.

Once the electronics control module determines that the post-nm state 412 is complete, it may proceed to a batch query 414, where the electronics control module may determine if there are more batches of goods to be treated with an antimicrobial treatment. If the electronics control module determines that the answer to the batch query 414 is YES, then the electronics control module may proceed back to running state 410 and proceed accordingly. If the electronics control module determines that the answer to the batch query 414 is NO, then the electronics control module may proceed back to idle state 408 and proceed accordingly. Other examples are possible.

For example, the electronics control module may determine that there are one or more errors in the system and take one or more responsive actions. For example, in response to determining that there are one or more errors in the system (e.g., one or more sensors need to be repaired, the cartridge does not have enough concentrated solution for a requested treatment cycle, etc.), the electronics control module may automatically revert back to setup 404 and proceed accordingly. In another example, the electronics control module may perform an error limit query 418, which may determine if the system is experiencing a numbers of errors and/or type of system error that are above a predetermined threshold. If the electronics control module determines that the answer to the error limit query 418 is NO, then the electronics control module may note that the system is in an error state 416 and proceed back to setup 404 and proceed accordingly. If the electronics control module determines that the answer to the error limit query 418 is YES, then the electronics control module may proceed to error lock state 420.

Once in error lock state 420, the system may not undertake any further treatment cycles and/or system and/or component diagnostics until the manufacturer, support entities, and/or other parties are apprised of the operational status of the system. In a further aspect, because the electronics control module may also be connected to one or more of other systems, once the system enters error lock state 420, the electronics control module may generate a report that contains the details of the last error it detected before entering the error lock state 420 and transmit this information to the user, manufacturer, support entities, and/or other parties. Other examples are possible.

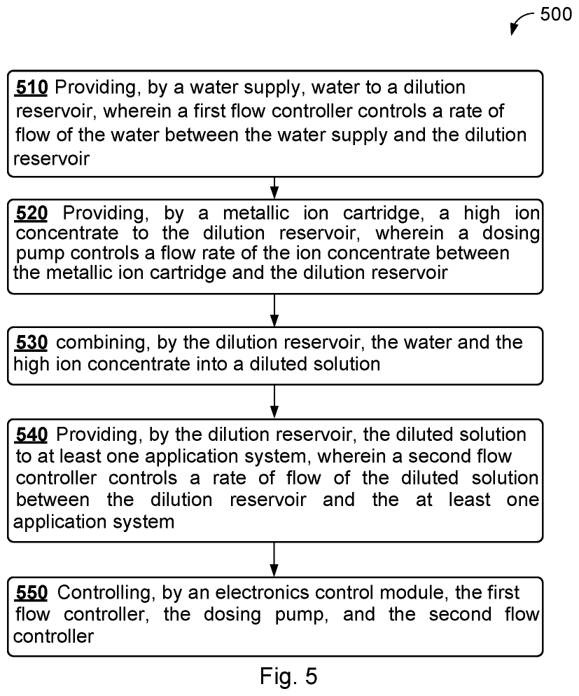

FIG. 5 is a flow chart illustrating an example embodiment. In FIG. 5, a method 500 for using a portable antimicrobial treatment system is described. The process illustrated by FIG. 5 may be carried out by a system, such as systems 100 and/or 200, and/or specific components thereof However, the process can be carried out by other types of devices or device subsystems.

At block 510, a water supply provides water to a dilution reservoir. A first flow controller controls a rate of flow of the water between the water supply and the dilution reservoir.

At block 520, a metallic ion cartridge provides a high ion concentrate to the dilution reservoir. A dosing pump controls a flow rate of the ion concentrate between the metallic ion cartridge and the dilution reservoir. In further example embodiments, the metallic ion cartridge also includes an RFID tag indicating an amount of the high ion concentrate therein.

At block 530, a dilution reservoir is used to combine the water and the high ion concentrate into a diluted solution.

At block 540, the dilution reservoir provides the diluted solution to at least one application system. A second flow controller controls a rate of flow of the diluted solution between the dilution reservoir and the at least one application system. In further example embodiments, the at least one application system is at least one textile treatment system. In further example embodiments, the at least one application system is at least one food product treatment system.

At block 550, an electronics control module controls the first flow controller, the dosing pump, and the second flow controller.

In further example embodiments, the method 500 for using a portable antimicrobial treatment system further comprises using a water quality sensor connected to the electronics control module to measure a quality of the water. In other example embodiments, the method 500 for using a portable antimicrobial treatment system further includes using a rotameter sensor connected to the electronics control module to measure the flowrate of the water. In other example embodiments, the method 500 for using a portable antimicrobial treatment system further includes using a pressure sensor connected to the electronics control module to measure a pressure of the water. In other example embodiments, the method 500 for using a portable antimicrobial treatment system further includes using a conductivity sensor connected to the electronics control module to measure a conductivity of the diluted solution. In still other example embodiments, the electronics control module is in communication with the RFID tag and is configured to update the indicated amount of the high ion concentrate after the metallic ion cartridge provides the high ion concentrate.

The singular forms of the articles “a,” “an,” and “the” include plural references unless the context clearly indicates otherwise. For example, the term “a compound” or “at least one compound” can include a plurality of compounds, including mixtures thereof.

Various aspects and embodiments have been disclosed herein, but other aspects and embodiments will certainly be apparent to those skilled in the art. Additionally, the various aspects and embodiments disclosed herein are provided for explanatory purposes and are not intended to be limiting, with the true scope being indicated by the following claims.

1. A portable antimicrobial treatment system comprising:
a water supply configured to provide water to an output of the water supply;
a first flow controller connected to the output of the water supply;
a metallic ion cartridge configured to provide a high ion concentrate to an output of the metallic ion cartridge;
a dosing pump to dispense the high ion concentrate and connected to the output of the metallic ion cartridge;
a dilution reservoir connected to the output of the dosing pump and to the first flow controller, wherein the dilution reservoir is configured to combine the water and the high ion concentrate into a diluted solution;
a second flow controller connected to an output of the dilution reservoir, wherein the second flow controller provides the diluted solution to at least one application system; and
an electronics control module connected to the first flow controller, the dosing pump, and the second flow controller.

2. The portable antimicrobial treatment system of claim 1, further comprising a water quality sensor connected to the electronics control module, wherein the water quality sensor is configured to measure a quality of the water.

3. The portable antimicrobial treatment system of claim 1, further comprising a rotameter sensor connected to the electronics control module, wherein the rotameter sensor is configured to measure a flowrate of the water.

4. The portable antimicrobial treatment system of claim 1, further comprising a pressure sensor connected to the electronics control module, wherein the pressure sensor is configured to measure a pressure of the water.

5. The portable antimicrobial treatment system of claim 1, further comprising a conductivity sensor connected to the electronics control module, wherein the conductivity sensor is configured to measure a conductivity of the diluted solution.

6. The portable antimicrobial treatment system of claim 1, wherein the at least one application system comprises at least one textile treatment system.

7. The portable antimicrobial treatment system of claim 1, wherein the at least one application system comprises at least one food product treatment system.

8. The portable antimicrobial treatment system of claim 1, wherein the metallic ion cartridge further comprises a radio frequency identification (RFID) tag indicating an amount of the high ion concentrate therein.

9. The portable antimicrobial treatment system of claim 8, wherein the electronics control module is in communication with the RFID tag and is configured to update the indicated amount of the high ion concentrate after the metallic ion cartridge provides the high ion concentrate.

10. A method for using a portable antimicrobial treatment system comprising:
providing, by a water supply, water to a dilution reservoir, wherein a first flow controller controls a rate of flow of the water between the water supply and the dilution reservoir;
providing, by a metallic ion cartridge, a high ion concentrate to the dilution reservoir, wherein a dosing pump controls a flow rate of the ion concentrate between the metallic ion cartridge and the dilution reservoir;
combining, by the dilution reservoir, the water and the high ion concentrate into a diluted solution;
providing, by the dilution reservoir, the diluted solution to at least one application system, wherein a second flow controller controls a rate of flow of the diluted solution between the dilution reservoir and the at least one application system; and
controlling, by an electronics control module, the first flow controller, the dosing pump, and the second flow controller.

11. The method of claim 10, wherein the method further comprises measuring, by a water quality sensor connected to the electronics control module, a quality of the water.

12. The method of claim 10, wherein the method further comprises measuring, by a rotameter sensor connected to the electronics control module, a flowrate of the water.

13. The method of claim 10, wherein the method further comprises measuring, by a pressure sensor connected to the electronics control module, a pressure of the water.

14. The method of claim 10, wherein the method further comprises measuring, by a conductivity sensor connected to the electronics control module, a conductivity of the diluted solution.

15. The method of claim 10, wherein the at least one application system comprises at least one textile treatment system.

16. The method of claim 10, wherein the at least one application system comprises at least one food product treatment system.

17. A non-transitory computer-readable medium, having stored thereon program instructions that, upon execution by an electronics control module, cause the electronics control module to perform a set of operations comprising:
providing, by a water supply, water to a dilution reservoir, wherein a first flow controller controls a rate of flow of the water between the water supply and the dilution reservoir;
providing, by a metallic ion cartridge, a high ion concentrate to the dilution reservoir, wherein a dosing pump controls a flow rate of the ion concentrate between the metallic ion cartridge and the dilution reservoir;
combining, by the dilution reservoir, the water and the high ion concentrate into a diluted solution;
providing, by the dilution reservoir, the diluted solution to at least one application system, wherein a second flow controller controls a rate of flow of the diluted solution between the dilution reservoir and the at least one application system; and
controlling, by an electronics control module, the first flow controller, the dosing pump, and the second flow controller.

18. The non-transitory computer-readable medium of claim 17, wherein the set of operations further comprise measuring, by a water quality sensor connected to the electronics control module, a quality of the water.

19. The non-transitory computer-readable medium of claim 17, wherein the set of operations further comprise measuring, by a rotameter sensor connected to the electronics control module, a flowrate of the water.

20. The non-transitory computer-readable medium of claim 17, wherein the set of operations further comprise measuring, by a pressure sensor connected to the electronics control module, a pressure of the water.
